(19) United States
(12) Patent Application Publication
(10) Pub. No.: US 2007/0005135 A1
(43) Pub. Date: Jan. 4, 2007

(54) Intraocular lens insertion plunger with low stimulus soft tip

(76) Inventors: Harish Makker, Mission Viejo, CA (US); Can B. Hu, Irvine, CA (US); Michael D. Lowery, Vista, CA (US)

Correspondence Address: Advanced Medical Optics, Inc.;Legal Department, 1700 E. St. Andrew Place, Santa Ana, CA, 92705, US

(21) Appl. No.: 11/173,337

(22) Filed: Jul. 1, 2005

(51) Int. Cl. A61F 2/16

(52) U.S. Cl. 623/6.12

(57) ABSTRACT

An intraocular lens (IOL) insertion apparatus for implanting IOLs through smaller incisions. The insertion apparatus includes an insertion cartridge that receives the IOL and cooperates with a handpiece. The cartridge includes a longitudinal lumen from a loading chamber to an open distal mouth that gradually narrows in dimension. A push rod having an extremely soft tip thereon urges the IOL through the cartridge and from the open distal mouth. The soft tip is made of low-stimulus material having a minimum ultimate elongation of 400%. Because of the extremely soft material of the tip, the outside diameter of the open distal mouth can be reduced to no more than 2.0 mm, enabling passage through incisions of 2.2 mm or less. The soft tip may be a thermoplastic elastomer having a relatively high elongation and relatively low modulus at elongations of 100-300%.

### FIELD OF THE INVENTION

The present invention relates generally to apparatus for inserting intraocular lenses (IOLs) into an eye. More particularly, the present invention relates to insertion apparatus having a hollow tube through which an IOL is pushed with a push rod having an extremely soft tip into an eye.

### BACKGROUND OF THE INVENTION

The human eye is susceptible to numerous disorders and diseases, a number of which attack the crystalline lens. For example, cataracts mar vision through cloudy or opaque discoloration of the lens of the eye. Cataracts often result in partial or complete blindness. If this is the case, the crystalline lens can be removed and replaced with an intraocular lens, or IOL.

An IOL is implanted in the eye not only as a replacement for the natural crystalline lens but also to alter the optical properties of (provide vision correction to) an eye in which the natural lens remains. IOLs often include an optically clear disk-like optic of about 6 mm in diameter, and preferably at least one flexible fixation member or haptic which extends radially outward from the optic and becomes affixed in the eye to secure the lens in position. Implantation of such IOLs into the eye involves making an incision through the cornea, sclera or the limbus between the cornea and sclera. It is advantageous, to reduce trauma and speed healing, to have an incision size as small as possible.

The optics may be constructed of rigid biocompatible materials such as polymethyl methacrylate (PMMA) or deformable materials such as silicone polymeric materials, acrylic polymeric materials, hydrogel polymeric materials, and the like. The deformable materials allow the IOL to be rolled or folded for insertion through smaller incisions into the eye. A substantial number of instruments have been proposed to aid in inserting such a foldable lens in the eye. In a popular apparatus, the optic begins in the shape of a taco and is pushed through an insertion cartridge so it is progressively rolled into a tubular shape to fit through the incision. Such an exemplary insertion system is disclosed in Makker et al., U.S. Pat. No. 5,942,277, the contents of which are expressly incorporated by reference herein.

The two primary deformable IOL materials are silicone and acrylic. Silicone IOLs are more pliable and can be folded into smaller tubes without unduly stressing the insertion cartridge or requiring excessive push force, which can violently expel the IOL from the cartridge. Acrylic lenses are indicated for some patients and are inserted in much the same way as silicone IOLs, although using larger bore cartridges to mitigate the problems caused by the lower flexibility of the acrylic. Because the cartridge bore is larger, the incision is also necessarily larger.

Despite the state of the art, IOL insertion apparatuses that enable IOLs to be inserted through smaller incisions are always beneficial.

### SUMMARY OF THE INVENTION

The present invention improves on earlier IOL insertion systems by enabling small incisions to be made, specifically incisions of 2.2 mm or less. To do this, the injection tube is desirably 2.0 mm or less in outside diameter. This, in turn, is enabled by a new soft tip for the insertion system push rod that is ultra-soft and easily deforms through the smaller injection tube. Various materials may be utilized for the soft tip, although a solid embodiment is defined by its ultimate elongation property, preferably coupled with a relatively low elastic modulus at specific elongation or strain values.

In accordance with one embodiment, a soft tip for the end of a push rod for inserting an intraocular lens through a tube is made of a solid material having a minimum ultimate elongation of 400%, and an elastic modulus of between 100 psi (689 kPa) and 310 psi (2137 kPa) at an elongation of 100%. Desirably, the soft tip material has an ultimate elongation of at least 780% and an elastic modulus of between 210 psi (1448 kPa) and 540 psi (3723 kPa) at an elongation of 300%. In accordance with a preferred soft tip material, the maximum hardness is 60 A Shore, and the tensile strength is at least 400 psi (2758 kPa). The material may be a thermoplastic elastomer, silicone, or other material that meets the performance criteria.

In accordance with another embodiment, the invention provides a soft tip for the end of a push rod for inserting an intraocular lens through a tube, wherein the soft tip is solid and made of a thermoplastic elastomer having a minimum ultimate elongation of 400%, preferably at least 780%. The thermoplastic elastomer desirably has an elastic modulus of between 100 psi (689 kPa) and 310 psi (2137 kPa) at an elongation of 100%, and between 210 psi (1448 kPa) and 540 psi (3723 kPa) at an elongation of 300%.

In accordance with a further aspect of the invention, an apparatus for inserting an intraocular lens into an eye is provided that comprises a cartridge having a load chamber for receiving an intraocular lens. The load chamber has an open proximal end and a distal end aligned with an injection tube having a tapered internal lumen that terminates at an open mouth with an outside diameter of no more than 2.0 mm. A push rod having a soft tip associated therewith is aligned with the cartridge such that, under urging of the push rod, the soft tip enters the proximal end of the load chamber and pushes distally on an intraocular lens therein. The soft tip is solid and formed of a material having a minimum ultimate elongation of 400% and an elastic modulus of between 100 psi (689 kPa) and 310 psi (2137 kPa) at an elongation of 100%. Preferably, the soft tip has a more rigid insert embedded therein which removably couples to a distal end of the push rod.

In accordance with a still further aspect, the invention provides a method for inserting an intraocular lens into an eye. The method includes forming an incision in a patient's cornea or sclera of no more than 2.2 mm. A cartridge is provided having a load chamber with an open proximal end and an open distal end aligned with an injection tube, the injection tube having a tapered internal lumen that terminates at an open mouth with an outside diameter of no more than 2.0 mm. The injection tube may have portions that are symmetrically or asymmetrically tapered. The method includes placing an intraocular lens in the load chamber, inserting a soft tip associated with a push rod into the proximal end of the cartridge, inserting the open mouth of the injection tube through the incision, and urging the intraocular lens from the load chamber through the tapered internal lumen. The intraocular lens is urged out of the open mouth of the injection tube and into the eye by the push rod and soft tip. In one embodiment, the mouth of the injection tube has an outside diameter of no more than 1.8 mm, preferably about 1.4 mm. The soft tip may be inflated, inflatable or solid. Desirably, a solid soft tip has an elastic modulus of between 100 psi (689 kPa) and 310 psi (2137 kPa) at an elongation of 100%, and between 210 psi (1448 kPa) and 540 psi (3723 kPa) at an elongation of 300%. The soft tip may have an ultimate elongation of at least 400%, preferably 780% or greater, and is desirably a thermoplastic elastomer.

In accordance with an alternative embodiment, the present invention provides an apparatus for inserting an intraocular lens into an eye comprising a cartridge having a load chamber for receiving an intraocular lens. The load chamber has an open proximal end and a distal end aligned with an injection tube having a tapered internal lumen that terminates at an open mouth with an outside diameter of no more than 2.0 mm. A push rod having an inflated soft tip associated therewith is aligned with the cartridge such that, under urging of the push rod, the soft tip enters the proximal end of the load chamber and pushes distally on an intraocular lens therein. The soft tip and push rod may not be coupled together. Desirably, the soft tip is under-inflated so as to deform as it travels through the tapered internal lumen of the injection tube.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

FIG. 1 is a front side view, in perspective, of an IOL insertion cartridge in accordance with the present invention with the load chamber in the open position;

FIG. 2 is a side view, in perspective, of the IOL insertion cartridge of FIG. 1 with the load chamber in the closed position;

FIG. 3 is a side view, partly in cross-section, of the IOL insertion cartridge positioned within an injector hand piece as taken along line 3-3 of FIG. 4;

FIG. 4 is a front side view, in perspective, of the IOL insertion cartridge of FIG. 2 loaded into a hand piece;

FIG. 5 is a somewhat schematic illustration showing the IOL insertion cartridge and hand piece of FIG. 3, with the hand piece partially in cross-section, being used to insert an IOL into an eye;

FIGS. 6A-6C are side cross-sectional views of the IOL insertion cartridge in the injector hand piece showing the advancement of a push rod and exemplary solid soft tip through the cartridge and injector tube lumens to urge an IOL from the injector tube;

FIGS. 7-9 are perspective views of exemplary shapes for the soft tip of the present invention and modes of connecting a push rod thereto; and

FIGS. 10A-10C are side cross-sectional views of the IOL insertion cartridge in the injector hand piece showing the advancement of a push rod and inflated or inflatable soft tip of the present invention.

### DESCRIPTION OF THE PREFERRED EMBODIMENTS

The present invention provides an improved IOL injection system that enables IOLs to be implanted through smaller incisions in the eye than has previously been possible. The limiting factor for the size of the incision is the outside diameter of the injection tube. Up to now, injection tubes down to about 2.2 mm in outside diameter have been available, which fit through incisions of about 2.8 mm or more. Assuming non-elastic tissue, the length L of the incision determines the maximum outside diameter OD of the injection tube using the formula: OD=2 L/π. However, some stretching of the incision can be anticipated and therefore a more realistic approximation is OD=0.8 L. The present invention reduces the size of the injection tube such that its outside diameter is less than 2.0 mm, preferably less than 1.8 mm, and most preferably about 1.4 mm. This enables introduction of IOLs through incisions of 2.5 mm or less, preferably 2.2 mm or less. Although the reduction in the incision length may seem small, there is an attendant reduction in healing time and complications that make any incremental decrease highly significant.

The invention may be incorporated into an injector system similar in kind to those described in the prior art, except for certain dimensions culminating in the smaller diameter distal end of the injection tube. Such prior art injector systems may include syringe-type plungers, plungers having screw-type advancement mechanisms, and the like. In addition, a push rod used to urge the IOL through the injection tube is equipped with an extremely soft tip having a low stimulus threshold for elongation. That is, the material of the soft tip has a very high ultimate elongation, the minimum of which is preferably 400%. The term “low stimulus” refers to the property of a material to easily deform from the application of a very low force. A number of materials are suitable for such an application, and the invention should not be considered limited to the specific materials described. Instead, the invention proposes a number of materials having certain properties that can be generally termed “low stimulus” or “high elongation”. To better understand the terminology used to describe the preferred materials, and those of the prior art, certain definitions must be spelled out.

Definitions:

Polymer, n: Any of numerous natural and synthetic compounds of usually high molecular weight consisting of up to millions of repeated linked units, each a relatively light and simple molecule. A chemical compound or mixture of compounds formed by polymerization and consisting essentially of repeating structural units.

Elastomer (e.g., silicone), n: Any of various polymers having the elastic properties of natural rubber. Any of various elastic materials that resemble rubber in that they resume their original shapes when a deforming force is removed, or that they can be stretched to many times their original length and bounce back into their original shape without permanent deformation.

Foam, n: Any of various light, porous, semirigid or spongy materials used for thermal insulation or shock absorption, as in packaging.

Plastic, n: Any of various organic compounds produced by polymerization, capable of being molded, extruded, cast into various shapes and films, or drawn into filaments used as textile fibers. Plastics are pliable; i.e., they can be shaped and molded easily. Plastics that become easier to mold and shape when they are hot, and melt when they get hot enough, are termed thermoplastics (see below). This is in contrast with cross-linked materials that do not melt, called thermosets (see below).

Resin, n: Any of numerous physically similar polymerized synthetics or chemically modified natural resins including thermoplastic materials such as polyvinyl, polystyrene, and polyethylene, and thermosetting materials such as polyesters, epoxies, and silicones, that are used with fillers, stabilizers, pigments, and other components to form plastics. Any of numerous physically similar polymerized synthetics or chemically modified natural resins including thermoplastic materials and thermosetting materials.

Thermoplastic, n: A thermoplastic resin, such as polystyrene or polyethylene. A material that softens when heated and hardens again when cooled [syn: thermoplastic resin]. A polymer that softens or melts on heating, and returns to its original condition when cooled to room temperature. Thermoplastic polymer chains are not cross-linked. Polystyrene is a thermoplastic. A classification for materials that can be made soft by the application of heat and harden upon cooling.

Thermoplastic, adj: Becoming soft when heated and hard when cooled. Having the property of softening or fusing when heated and of hardening and returning to its original condition as it is cooled to room temperature. Thermoplastic materials can be remelted and cooled time after time without undergoing any appreciable chemical change [ant: thermosetting].

Thermoset, n: A classification for materials that become hardened or cured by the application of heat or radiation, usually associated with a cross-linking reaction. In many cases, it is necessary to add “curing” agents such as organic peroxides or other compounds. Once cured, thermoset materials cannot be remelted and reprocessed.

Thermoset, adj: Having the property of becoming permanently hard when heated or cured.

Silicone, n: Any of many polymers made up of alternating oxygen and silicon atoms combined with other organic groups. Silicone can be an oil, grease, gel or plastic, but all forms are resistant to heat and water, and will not conduct electricity. Silicone can copolymerize with other materials such as urethane to form a thermoplastic material with properties similar to a typical thermoplastic elastomer. Silicone is a typical example of thermoset material which can be cured by peroxide, moisture, platinum or other compounds.

Rubber, n: A yellowish, amorphous, elastic material obtained from the milky sap or latex of various tropical plants, especially the rubber tree, and vulcanized or cross-linked, pigmented, finished, and modified into products such as electric insulation, elastic bands and belts, tires, and containers. Cross-linked rubber has covalent links between the different polymer chains, joining them all into a single networked molecule. When the polymer chains are joined together like this, it is even harder to pull them out of their original positions, and so the rubber bounces back even better when stretched. Because the polymer chains are tied together, when the rubber gets hot, they cannot flow past each other, nor around each other. This is why it does not melt.

Sponge, n: A piece of absorbent porous material, such as cellulose, plastic, or rubber, used especially for washing and cleaning.

Glass transition temperature (Tg), n: When an amorphous polymer is cooled below this temperature, it becomes hard and brittle, like glass. Some polymers are used above their glass transition temperatures, and some are used below. Hard plastics like polystyrene and poly(methyl methacrylate), are used below their glass transition temperatures; that is in their glassy state. Their Tg's are well above room temperature, both at around 100° C. Rubber elastomers like polyisoprene and polyisobutylene, are used above their Tg's, that is, in the rubbery state, where they are soft and flexible.

Amorphous polymers, n: polymers whose chains are primarily not arranged in ordered crystals, but are strewn around in random order, even though they are in the solid state. Not all amorphous polymers are elastomers. Some are thermoplastics. Whether an amorphous polymer is a thermoplastic or an elastomer depends on its glass transition temperature. If an amorphous polymer has a T_{g} below room temperature, that polymer will be an elastomer, because it is soft and rubbery at room temperature. If an amorphous polymer has a T_{g} above room temperature, it will be a thermoplastic, because it is hard and glassy at room temperature. So a general rule of thumb is that for amorphous polymers, elastomers have low T_{g}'s and thermoplastics have high T_{g}'s. (This only works for amorphous polymers, and not for crystalline polymers.)

Block copolymer: A copolymer is a polymer made from more than one kind of monomer, that is, made out of two or more co-monomers. A block copolymer is a copolymer in which the co-monomers are separated into long sections of the polymer backbone chain. Each of these sections, called blocks, looks sort of like a homopolymer.

Ionomer: A kind of copolymer. They are copolymers in which a small portion of the repeat units have ionic pendant groups attached to them. Normally the polymer backbone chain will be nonpolar. The nonpolar polymer backbone chains will group together, and the polar ionic pendant groups will cluster together. The clusters of ionic groups cannot separate themselves completely from the nonpolar backbone chains and serve to tie the backbone chains together, just like a normal cross-link would.

Cross-linked polymers, n: Cross-linked polymers are usually molded and shaped before they are cross-linked. Once cross-linking has taken place, usually at high temperature, the object can no longer be shaped. Because heat usually causes the cross-linking which makes the shape permanent, these materials are called thermosets. This name distinguishes them from thermoplastics, which are not cross-linked and can be reshaped once molded. Normal cross-linked polymers cannot be recycled because they do not melt. They do not melt because the cross-links tie all the polymer chains together, making it impossible for the material to flow.

Thermoplastic elastomer (TPE), n: TPEs have the properties of elastomers with the processing benefits of thermoplastics. A tough, electrically insulating elastomer, with many of the physical properties of vulcanized rubbers but which, unlike conventional vulcanized rubbers, can be processed as a thermoplastic material. Most are two-phase systems that have hard and soft phases. Applications include adhesives, coatings for wire and cable, automotive bumpers, footwear, medical devices, polymer modification, and vibration dampers. A polymer blend or compound which above its melt temperature, exhibits a thermoplastic character that enables it to be shaped into a fabricated article and which, within its design temperature range, possesses elastomeric behavior without cross-linking during fabrication. This process is reversible and the products can be reprocessed and remolded. (Synonyms: thermoplastic rubber, TPE, TPO rubber).

The idea behind thermoplastic elastomers (TPEs) is the notion of a reversible cross-link. Normal cross-links are covalent, chemically bonding the polymer chains together into one molecule. The reversible cross-link uses non-covalent, or secondary interactions between the polymer chains to bind them together. These interactions include hydrogen bonding and ionic bonding. The beauty of using noncovalent interactions to form cross-links is that when the material is heated, the cross-links are broken. This allows the material to be processed, and most importantly, recycled. When it cools again, the cross-links reform. Two approaches have been tried, ionomers and block copolymers.

Now that certain foundational definitions have been provided, an understanding of the present invention should become easier. As mentioned above, the invention ultimately results in a smaller incision in the cornea, sclera or limbus between the cornea and sclera of the eye to inject an IOL therethrough. An exemplary system for injecting an IOL into the eye will now be described. It should be understood, however, that any system culminating at its distal end in a tube which passes through an incision in the eye and through which an IOL is expelled may benefit from various aspects of the present invention. Therefore, the IOL injector and insertion cartridge should be viewed as representative of the entire array of such systems.

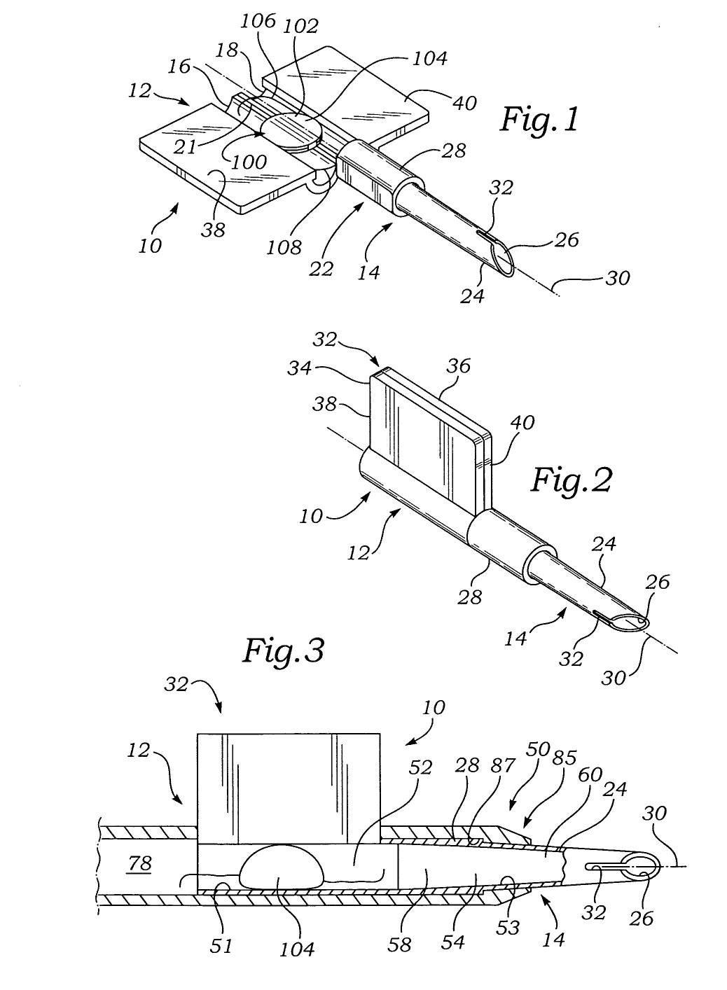

FIGS. 1-3 illustrate an IOL inserter cartridge 10 of the present invention. The body of the inserter cartridge 10 is an integrally formed, for example, molded, unit made primarily of polypropylene. A load chamber 12 includes a partial tubular first member 16 and a partial tubular second member 18 which are secured or joined together and are hingably moveable relative to each other along line 21 parallel to the longitudinal axis 30 of the cartridge 10.

An injection tube 14 includes a proximal end portion 22, a distal end portion 24 and an open distal mouth 26. A reinforcing collar 28 is coincidental with the proximal end portion 22 of injection tube 14. Open distal mouth 26 is beveled at an angle of about 45° relative to the longitudinal axis 30 of the cartridge 10. Injection tube 14 may include a through slot 32 which extends from the open distal mouth 26 distally and terminates prior to the proximal end portion 22 of injection tube 14. Through slot 32 is elongated in a direction parallel to the longitudinal axis 30 of the cartridge 10.

As shown in FIG. 1, the cartridge 10 is in the opened position. In contrast, in FIG. 2, the cartridge 10 is shown in the closed position. In the closed position, the load chamber 12 includes a top 32 which is a combination of top surfaces 34 and 36 of first wing 38 and second wing 40, respectively, of first member 16 and second member 18. First and second wings 38 and 40 are effective for a user of the cartridge 10 to hold and manipulate the cartridge 10 while using it, as described hereinafter.

The cartridge 10 is described in more detail with reference to FIG. 3, which shows the inserter in combination with a distal portion 50 of a hand piece. When used in combination with the hand piece, the load chamber 12 of the cartridge 10 is in the closed position. With the load chamber 12 in the closed position, and the top 32 being the uppermost portion of the load chamber, the open distal mouth 26 of injection tube 14 is beveled at an angle of 45° relative to the longitudinal axis 30 of the cartridge 10 so that the open distal mouth is generally right facing (when the inserter is viewed from above). In addition, if present, through slot 32 intersects the open distal mouth 26 at the proximal most portion of the open distal mouth, as shown in FIGS. 1-3.

Still with reference to FIG. 3, the load chamber 12 includes an interior wall 51 that defines a first lumen 52 elongated in a direction parallel to the longitudinal axis 30 of the cartridge 10. Injection tube 14 includes a tapering interior wall 53 defining a distally tapering second lumen 54 (e.g., the taper may be symmetrical or asymmetrical in one or more cross-sectional dimensions). The average cross-sectional area of second lumen 54 transverse to the longitudinal axis 30 is smaller than or reduced in at least one cross-sectional dimension relative to the average cross-sectional area of the first lumen 52.

The first lumen 52 is aligned with the second lumen 54 so that a folded IOL in the first lumen can be passed directly from the first lumen into the second lumen. The taper of proximal portion 58 of second lumen 54 is more severe than the slight taper which exists in the distal portion 60 of the second lumen. The more severe taper in the proximal portion 58 is effective to further fold the IOL as the IOL is passed into the second lumen 54. This further folding is advantageous because the further folded IOL can be inserted into the eye through a smaller incision.

Enhanced lubricity resulting from a component incorporated into the material of the cartridge 10 or coated (i.e., covalently bonded or otherwise) on the material of cartridge 10 facilitates this further folding so that a reduced amount of force is required. Another benefit is that the degree of folding of the IOL may be increased so that ultimately the IOL can be inserted through an even smaller incision. The lubricity-enhancing component also advantageously reduces the risk of tearing and/or otherwise damaging the IOL as the IOL is passed through the first lumen 52 and second lumen 54.

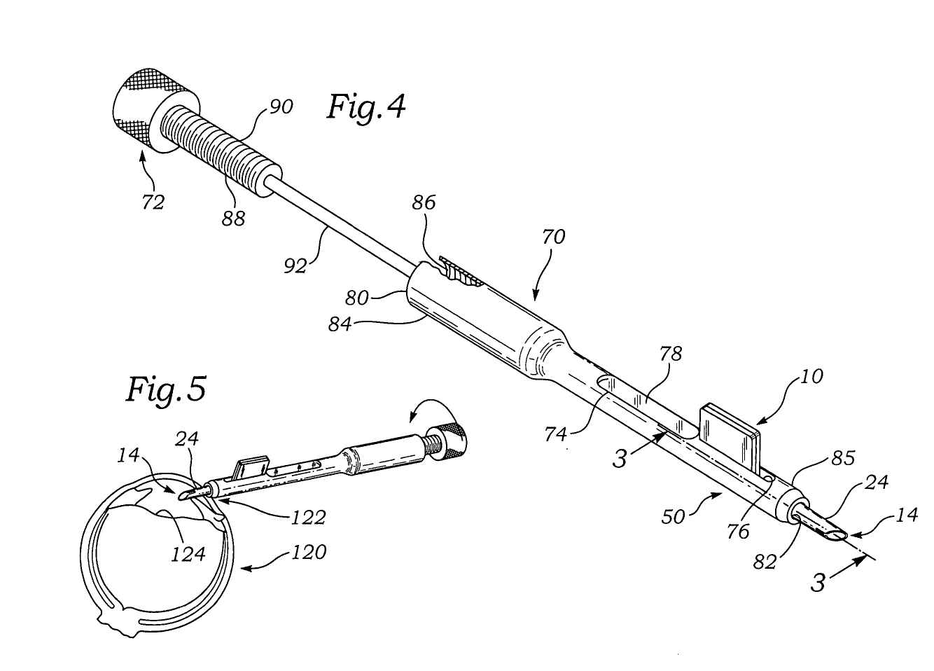

With reference to FIG. 4, the cartridge 10 is shown in combination with hand piece 70 and push rod 72. Hand piece 70 includes a relatively large, elongated first through opening 74 and a relatively small, elongated second through opening 76. Hand piece 70 includes a through bore 78 which extends from the proximal end 80 to the distal end 82 of the hand piece. The proximal end portion 84 of hand piece 70 includes threads 86 which are adapted to engage and mate with threads 88 of the proximal segment 90 of push rod 72. Alternately, a syringe style inserter may be used in which a plunger is depressed without rotation to advance the plunger. Shaft 92 of push rod 72 is aligned and sized to pass through bore 78, first lumen 52, second lumen 54 and out of open distal mouth 26. Hand piece 70 and push rod 72 are typically made of metal, such as titanium, surgical grade stainless steel or similar expedients, although plastics and other materials may be used (e.g., polypropylene). As will be described below, an extremely soft tip on the distal end of the shaft 92 actually contacts and urges the IOL through the cartridge 10.

The cartridge 10 is operated and functions as follows. When it is desired to load an IOL into the cartridge 10, the inserter is placed, for example, manually placed, in a configuration as shown in FIG. 1. With load chamber 12 in the opened position, an IOL, such as is shown generally at 100, is placed, for example, using forceps, in between first and second members 16 and 18. This placement is such that the anterior face 102 of optic 104 faces upwardly, as shown in FIG. 1. The optic 104 is made of a material such as silicone or acrylic with a glass transition temperature that is substantially lower than ambient temperature, and desirably having a T_{g} of 10° C. or less. For three-piece IOLs, fillet haptics or fixation members 106 and 108 of IOL 100 are located as shown so that the fixation members are located generally parallel to, rather than transverse to, the longitudinal axis 30. For one-piece IOLs, the haptics will be tucked inside the folded optic 104.

With IOL 100 placed as shown in FIG. 1, first and second members 16 and 18 are hingeably moved relative to each other, for example, by manually bringing first and second wings 38 and 40 together, to place the load chamber 12 in the closed position, as shown in FIG. 2. With load chamber 12 in the closed position, IOL 100 is in a folded state, that is optic 104 is folded. The relative movement of first and second members 16 and 18 to move the load chamber from the open position to the closed position is effective to fold the optic 104. The folded IOL 100 is now located in the first lumen 52 as seen in the cross-section of FIG. 3.

With the cartridge 10 configured as shown in FIG. 3 and folded IOL optic 104 located in first lumen 52, the cartridge 10 is placed in association with hand piece 70, as shown in FIG. 4. In this configuration, the distal end portion 24 of injection tube 14 extends distally beyond the distal end 82 of hand piece 70. As shown in FIG. 3, the distal portion 85 of hand piece 70 includes an inner wall 87 that is configured to receive reinforcing collar 28 in abutting relation.

Referring now to FIG. 5, the IOL 100 is to be placed in eye 120 into an area formerly occupied by the natural lens of the eye. FIG. 5 shows the cornea 122 having an incision through which the distal end portion 24 of injection tube 14 is passed. Alternately, the incision can be made through the sclera or limbus. Distal end portion 24 has a sufficiently small cross-section to pass into the eye 122 through an incision in the sclera 122 (or cornea) of no more than 2.2 mm in length.

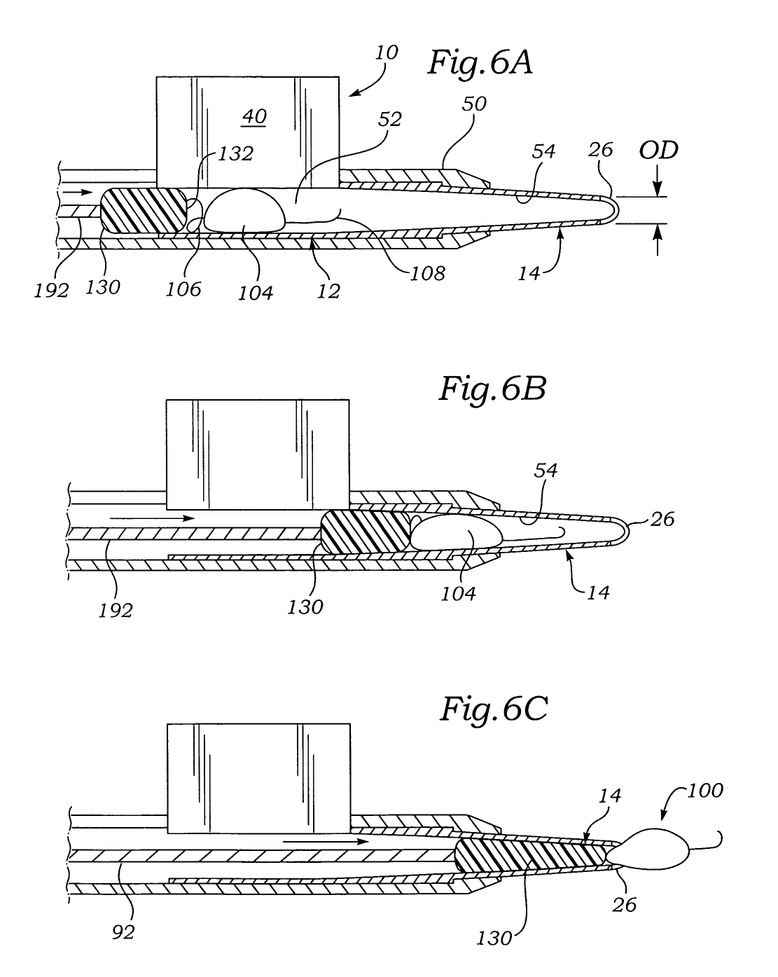

With the cartridge 10 positioned within the hand piece 70, the push rod 72 is placed into the through bore 78 of the hand piece starting at the proximal end 80. As threads 88 come in contact with and engage threads 86, the push rod 72 is rotated, as shown in FIG. 5, so as to thread the push rod onto the proximal end portion 84 of hand piece 70. The push rod 72 and the cartridge 10 are aligned such that the shaft 92 and soft tip thereon enters the proximal end of the load chamber 12 and pushes distally on the IOL 100 therein. By gradually moving shaft 92 through bore 78 of hand piece 70, the folded IOL 100 is urged by the soft tip to move from first lumen 52 into second lumen 56, through open distal mouth 26 and into the eye. The exemplary process by which the IOL is urged through the cartridge 10 is described and shown in more detail below with respect to FIGS. 6A-6C.

The injection tube 14 is manipulated until it is positioned so that IOL 100 can be properly positioned in the eye 122, that is in the anterior chamber, the posterior chamber, the capsular bag 124 or in the sulcus, after being released. Thus, the surgeon is able to controllably position the distal end portion 24 of injection tube 14, with the IOL 100 in the first lumen 52 of the load chamber 12. Once the distal end portion 24 is so positioned, the shaft 92 is urged distally, by rotating (threading) push rod 72 onto hand piece 70, to pass the IOL 100 into and through the second lumen 54, through the open distal mouth 26 of injection tube 14 and into the eye 120.

The anterior face 102 of IOL 100 faces generally forwardly in the eye 120 as the IOL is released from the cartridge 10. In other words, the IOL 100 passes through first lumen 52, second lumen 54 and open distal mouth 26 and into eye 120 without flipping or otherwise becoming mispositioned. Only a relatively small amount of, if any, post-insertion re-positioning is needed to properly position IOL 100 in eye 120.

After the IOL 100 has been inserted into the eye, the shaft 92 is moved proximally into the injection tube 14 and the distal end portion 24 of the injection tube is removed from the eye. If needed, the IOL 100 can be repositioned in the eye by a small, bent needle or similar tool inserted into the same incision.

Once the IOL 100 is properly positioned in eye 120 and the cartridge 10 is withdrawn from the eye, the incision in the cornea, sclera or limbus may be mended, for example, using conventional techniques. After use, the cartridge 10 is preferably disposed. Hand piece 70 and push rod 72 can be reused, after sterilization/disinfection. The soft tip is typically also disposed.

With reference now to FIGS. 6A-6C, a first exemplary embodiment of a soft tip 130 provided on the end of the push rod shaft 92 is shown in a series of views urging an IOL through an injection tube 14. The use of the soft tip 130 is described in the context of the above-described inserter cartridge 10, and therefore like numbers will be used. It should, however, be repeated that this system for injecting an IOL into an eye is exemplary only.

The soft tip 130 comprises a solid block of material that is desirably cylindrical having an axis coincident with the axis of the shaft 92. Alternately, the tip may be offset or asymmetrically shaped relative to the axis of the shaft, such that the tip pushes preferentially against one or more sides of the cartridge. To help the soft tip 130 enter the proximal end of the load chamber 12, its distal face 132 may be rounded or provided with a circular chamfer as shown. Of course, a lubricating material typically provided within the load chamber 12 also facilitates advancement of the soft tip 130. In addition or alternately, a lubricious material may be incorporated into or coated on the soft tip 130. FIG. 6A shows the soft tip 130 after it has partially entered the load chamber 112, and in particular the first lumen 52. The distal face 132 forms a pressing surface to urge the IOL through the inserter cartridge 10. If the IOL has three pieces, an optic 104 and two haptics 106, 108, the distal face 132 first contacts the trailing haptic 106 as shown.

As the push rod shaft 92 displaces further in a distal direction, eventually the distal face 132 of the soft tip 130 contacts the optic 14 and urges the entire IOL into the tapered second lumen 54 of the injection tube 14 as shown in FIG. 6B. The outside diameter of the soft tip 130 is substantially the same as the inside diameter of the first lumen 52, and therefore as the soft tip advances into the narrowing second lumen 54 it begins to deform, as shown. Specifically, the soft tip 130 gradually lengthens as it is forced into the smaller diameter second lumen 54. As will be described below, the material of the soft tip 130 is a type that deforms extremely easily upon application of a very low stimulus, in this case the constriction imposed thereon by the tapered second lumen 54. Some of the deformation of the soft tip 130 will undoubtedly occur in a proximal direction around the shaft 92, but the majority appears as a distal lengthening within the second lumen 54.

Ultimately, FIG. 6C shows the push rod shaft 92 advanced to a point at which the soft tip 130 has lengthened considerably within the tapered second lumen 54 and in a position that expels the IOL 100 from the distal mouth 26 of the injection tube 14. Because of the high elongation property of the material of the soft tip 130, it does not apply excessive outward radial pressure on the injection tube 14, but instead deforms by lengthening. In one preferred material embodiment, the soft tip 130 possesses a minimum ultimate elongation of 400%. This is a significant improvement over earlier soft tips which did not have the same elongation properties, and therefore instead of deforming they would be simply placed under great pressure eventually exerting that pressure outward and damaging the injection tube. Another possible scenario is that when the soft tips of the prior art are squeezed through too small of an injection tube they may damage the IOL.

Although the soft tip 130 has sufficient elongation properties to deform into the shape shown in FIG. 6C, it must of course not be completely deformable; it must have some elastic modulus under various amounts of stress. Otherwise, the soft tip 130 would not have sufficient integrity to maintain a distal force on the IOL, but instead might deform around the IOL or backward around the push rod as the reaction forces increase from the narrowing second lumen 54. Therefore, the soft tip 130 possesses very high elongation properties, and retains a relatively low elastic modulus at various states of elongation. In one preferred material embodiment, the soft tip 130 possesses an elastic modulus of between 100-200 psi (689-1448 kPa) at elongations of between 100-300%.

Certain thermoplastic elastomer materials are suitable for the soft tip 130, and specifics for three are provided in Table I below, with data supplied by GLS Corporation of McHenry, Ill. These data may be viewed online at http://www.glscorp.com/home.html.

TABLE I
Suitable Soft Tip Materials (from GLS Corporation)

| | | Tensile | 100% | 300% | | Tear |
|---|---|---|---|---|---|---|
| | Shore | Strength, | modulus, | modulus, | Percent | strength, |
| | Hardness, | psi | psi | psi | elongation | pli |
| GLS product | 10 sec delay | (kPa) | (kPa) | (kPa) | at break | (kN/m) |
| Versaflex ® CL30 | 30 A | 960 | 100 | 210 | 780 | 110 |
| | | (6619) | (689) | (1448) | | (19) |
| Dynaflex ® | 30 A | 400 | 100 | 230 | 640 | 90 |
| G7930-9001-02 | | (2758) | (689) | (1586) | | (16) |
| Dynaflex ® | 58 A | 1160 | 310 | 540 | 690 | 180 |
| G2703-1000-00 | | (7998) | (2137) | (3723) | | (32) |

These thermoplastic elastomer materials have been tested for use as the soft tip 130. The first-listed, Versaflex® CL30, seems to perform the best, with the second, Dynaflex® G7930-9001-02 performing well, and the third, Dynaflex® G2703-1000-00 performing adequately. All three of these materials have a relatively high elongation percent at break (i.e., ultimate elongation) greater than 600%. At a minimum, however, the soft tip 130 of the present invention should have an ultimate elongation of 400%, and preferably the ultimate elongation is 780% or more. Moreover, it is the low stimulus property of the soft tip materials during use that render them capable of deforming as they pass through the ever-narrowing injection tube lumen. At elongations of 100%, the thermoplastic elastomer materials tested had a minimum elastic modulus of 100 psi (689 kPa), and a maximum elastic modulus of 310 psi (2137 kPa). At 300% elongation the three materials tested had a minimum elastic modulus of 210 psi (1448 kPa), and a maximum elastic modulus of 540 psi (3723 kPa). Preferably, the material of the soft tip 130 has a relatively low elastic modulus that ranges between 100-210 psi (689-1448 kPa) at elongations of between 100-300%.

Moreover, certain other physical properties are also believed to be important. Although not willing to be bound by any particular formulation of physical properties, the performance of the soft tips of the present invention seems to depend upon a combination of factors, namely, hardness, tensile strength, modulus at 100%, modulus at 300%, and percent ultimate elongation of the material. Taking data from the materials tested, the hardness should be a maximum of 60 A Shore, desirably within the range of 30 A to 58 A Shore and more desirably less than 40 A Shore, and the tensile strength should be at least 400 psi (2758 kPa), more preferably between 400-1160 psi (2758-7998 kPa).

The materials tested and listed in Table I above were obtained from GLS Corporation of McHenry, Ill. The TPE materials offered by GLC come in the following classes: Styrenic block copolymers, thermoplastic vulcanizates, and thermoplastic polyurethanes. GLS trade names for potentially useful materials include Kraton, DYNAflex, VERSAflex, VERSalloy, and VERSollan.

It should be noted that although the preferred materials are thermoplastic elastomers, it is contemplated that other materials having similar physical response characteristics may be utilized. Indeed, the soft tip can be either a thermoplastic elastomer or a thermoset elastomer. Further, it is possible that certain formulations of silicone or silicone copolymer (“a silicone”) may be suitable in that they have the high elongation property coupled with the relatively low modulus values at 100% and 300% elongations. Once again, it is the low stimulus property of the soft tip materials that permit them to deform as they pass through the tapered injection tube lumen.

Moreover, although the preferred soft tip 130 described above is solid, meaning it has a primarily continuous and contiguous physical makeup across any section therethrough, other configurations such as the inflated or inflatable soft tip described below may behave in a manner analogous to a solid with the desired high elongation property, and therefore function adequately.

The physical characteristic of ultimate elongation of the material most distinguishes the soft tip materials of the present invention with those of the prior art. For example, U.S. Pat. No. 6,162,229 to Feingold, et al. describes an IOL injector having a deformable plunger tip made of a material such as an “elastomer (e.g., silicone), deformable plastic, deformable thermoplastic, rubber, foam, sponge, COLLAMER made by STAAR Surgical, or other suitable resiliently deformable material.” There are no further discussions of the specific properties of these various materials, other than that they are deformable through an injector cartridge. Also, there is no mention of the size of the tip of the injector cartridge, nor of the size of the incision through which the tip may be inserted.

COLLAMER is a proprietary collagen/polymer material similar to silicone in physical properties and used by STAAR Surgical of Monrovia, Calif. in its intraocular lenses. On the STAAR Surgical web site (www.staar.com) IOLs made with COLLAMER or silicone are described. Silicone differs in ultimate elongation percent depending on formulation and preparation, and no data is provided for the STAAR material. The following injector cartridges are also described on the STAAR Surgical web site (www.staar.com). It is important to note that the minimum size of incision is 2.8 mm, and therefore applicants presume that the silicone used is not the same as described herein and cannot pass through the very small injector tubes as disclosed in the present application.

AQ Cartridge

The MICRO-TIP CARTRIDGE® contains a 45 degree beveled edge and two slits at the cartridge tip. This cartridge is a mini-wing style and can be inserted through an incision size of 2.8 mm. This cartridge has been designed to accommodate STAAR three-piece style lenses.

MTC-60c Cartridge

The MICRO-TIP CARTRIDGE® contains a 60 degree beveled edge and no slits. This cartridge is a mini-wing style and can be inserted through an incision size of 2.8 mm. This cartridge has been designed to accommodate STAAR plate haptic lenses.

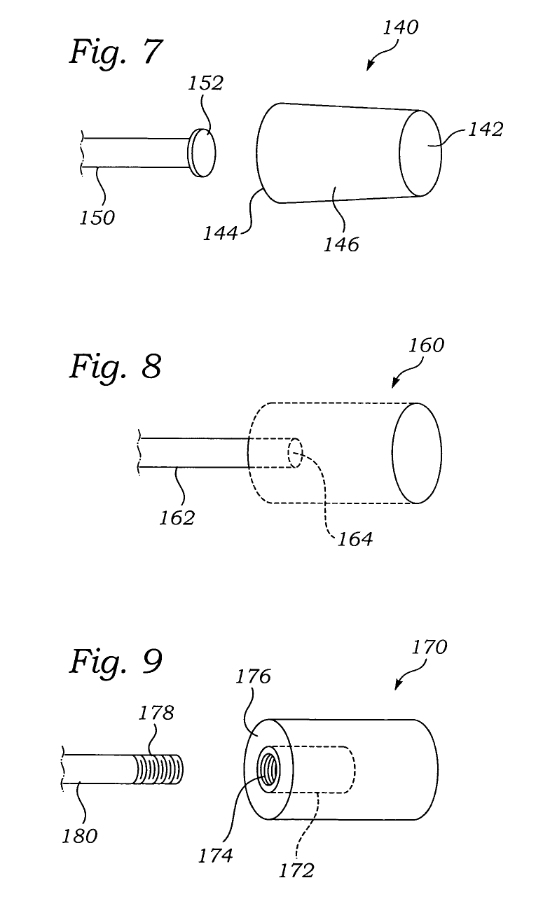

With reference now to FIGS. 7-9, various configurations of solid soft tips for use in the present invention are disclosed. In FIG. 7, a soft tip 140 made of any of the materials described above has a distal face 142, a proximal face 144, and a partial conical outer surface 146 therebetween. In other words, the distal face 142 has a smaller diameter than the proximal face 144. A push rod 150 is seen separated from the proximal face 144 and having a distal faceplate 152 thereon. The faceplate 152 is adapted to contact and push on the proximal face 144 of the soft tip 140, but is not otherwise coupled thereto. The faceplate 152 increases the surface area of the push rod 150 to apply a more widely distributed push force to the soft tip 140.

FIG. 8 illustrates a cylindrical soft tip 160 made of any of the materials described above. A push rod 162 is shown attached to the soft tip 160 by embedding a distal end 164 therein. The distal end 164 may fit tightly within a predrilled cavity in a proximal face of the soft tip 160, or may be secured more tightly therein using adhesives or the like. Although not shown, the distal end 164 may be shaped so as to snap fit or otherwise mate with a similarly-shaped cavity in the soft tip 160 for a more secure coupling. Such a rod/tip configuration is seen in Makker et al., U.S. Pat. No. 6,254,607, the contents of which are expressly incorporated by reference herein.

Finally, FIG. 9 illustrates a cylindrical soft tip 170 having a more rigid tubular insert 172 embedded therein. A threaded bore 174 of the insert 172 opens to the proximal face 176 of the soft tip 170. The threaded bore 174 receives a threaded portion 178 of a push rod 180. In this manner, the benefits of the soft tip material can be realized while the connection between the push rod 180 and soft tip 170 is enhanced. Furthermore, this enables the soft tip 170 to be easily replaced between uses.

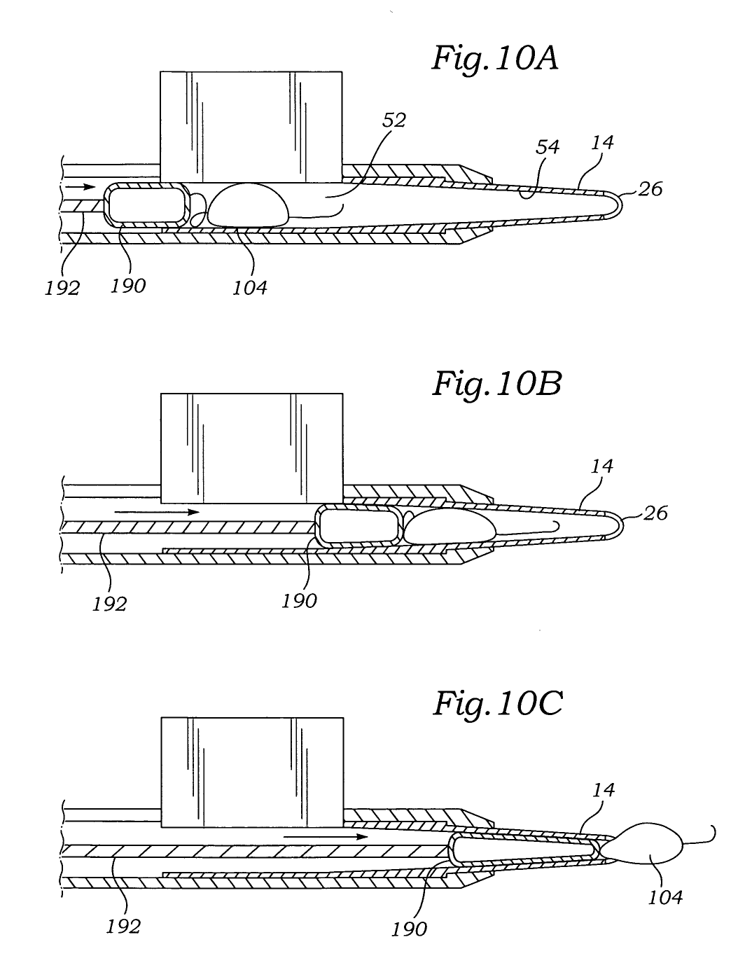

With reference now to FIGS. 10A-10C, an alternative embodiment of an inflatable soft tip 190 provided on the end of the push rod shaft 192 is shown in a series of views urging an IOL through an injection tube 14. The use of the soft tip 130 is described in the context of the above-described inserter cartridge 10, and therefore like numbers will be used.

The soft tip 190 comprises an inflatable or inflated member of latex or silicone, for example, that is desirably cylindrical and has an axis coincident with the axis of the shaft 192. To help the soft tip 190 enter the proximal end of the load chamber 12, its distal face may be rounded as shown. FIG. 10A shows the soft tip 190 after it has partially entered the load chamber 112, and in particular the first lumen 52. The distal face urges the IOL through the inserter cartridge 10.

As the push rod shaft 192 displaces further in a distal direction, eventually the distal face of the soft tip 190 contacts the optic 104 and urges the entire IOL into the tapered second lumen 54 of the injection tube 14 as shown in FIG. 10B. The outside diameter of the soft tip 190 is substantially the same as the inside diameter of the first lumen 52, and therefore as the soft tip advances into the narrowing second lumen 54 it begins to deform, as shown. Specifically, the soft tip 190 gradually lengthens as it is forced into the smaller diameter second lumen 54. To facilitate this deformation, the soft tip 190 is desirably under-inflated and therefore may be squeezed into the elongated shape. Some of the deformation of the soft tip 190 will undoubtedly occur in a proximal direction around the shaft 192, but the majority appears as a distal lengthening within the second lumen 54.

Ultimately, FIG. 10C shows the push rod shaft 192 advanced to a point at which the soft tip 190 has lengthened considerably within the tapered second lumen 54 and in a position that expels the IOL 100 from the distal mouth 26 of the injection tube 14. Because of the high elongation property of the material of the under inflated soft tip 190, it does not apply excessive outward radial pressure on the injection tube 14, but instead deforms by lengthening. In one preferred embodiment, the soft tip 190 is inflated to a degree such that it responds in a way that mimics a solid tip with a minimum ultimate elongation of 400%.

It will also be appreciated by those of skill in the relevant art that various changes may be made to the examples and embodiments of the invention described in this provisional application, without departing from the intended scope of the invention. The particular embodiments of the invention described herein are thus to be understood as examples of the broader inventive concept disclosed in this application.

What is claimed is:

1. A soft tip for the end of a push rod for inserting an intraocular lens through a tube, the soft tip being made of a solid material having a minimum ultimate elongation of 400%, and an elastic modulus of between 100 psi (689 kPa) and 310 psi (2137 kPa) at an elongation of 100%.

2. The soft tip of claim 1, wherein the soft tip material has an ultimate elongation of at least 780%.

3. The soft tip of claim 1, wherein the soft tip material has an elastic modulus of between 210 psi (1448 kPa) and 540 psi (3723 kPa) at an elongation of 300%.

4. The soft tip of claim 1, wherein the soft tip material has a maximum hardness of 60 A Shore.

5. The soft tip of claim 1, wherein the soft tip material has a tensile strength of at least 400 psi (2758 kPa).

6. The soft tip of claim 1, wherein the soft tip is a thermoplastic elastomer.

7. The soft tip of claim 1, wherein the soft tip is a silicone.

8. A soft tip for the end of a push rod for inserting an intraocular lens through a tube, the soft tip being solid and made of a thermoplastic elastomer having a minimum ultimate elongation of 400%.

9. The soft tip of claim 8, wherein the thermoplastic elastomer has an ultimate elongation of at least 780%.

10. The soft tip of claim 8, wherein the thermoplastic elastomer has an elastic modulus of between 100 psi (689 kPa) and 3.10 psi (2137 kPa) at an elongation of 100%.

11. The soft tip of claim 8, wherein the thermoplastic elastomer has an elastic modulus of between 210 psi (1448 kPa) and 540 psi (3723 kPa) at an elongation of 300%.

12. The soft tip of claim 8, wherein the thermoplastic elastomer has a maximum hardness of 60 A.

13. The soft tip of claim 8, wherein the thermoplastic elastomer has a tensile strength of at least 400 psi (2758 kPa).

14. An apparatus for inserting an intraocular lens into an eye, comprising:
a cartridge with a load chamber for receiving an intraocular lens, the load chamber having an open proximal end and a distal end aligned with an injection tube having a tapered internal lumen that terminates at an open mouth, the mouth having an outside diameter of no more than 2.0 mm; and
a push rod having a soft tip associated therewith, the push rod and the cartridge being aligned such that, under urging of the push rod, the soft tip enters the proximal end of the load chamber and pushes distally on an intraocular lens therein, the soft tip being solid and formed of a material having a minimum ultimate elongation of 400% and an elastic modulus of between 100 psi (689 kPa) and 310 psi (2137 kPa) at an elongation of 100%.

15. The apparatus of claim 14, wherein the soft tip material has an elastic modulus of between 210 psi (1448 kPa) and 540 psi (3723 kPa) at an elongation of 300%.

16. The apparatus of claim 14, wherein the soft tip material has an ultimate elongation of at least 780%.

17. The apparatus of claim 14, wherein the soft tip material has a maximum hardness of 60 A.

18. The apparatus of claim 14; wherein the soft tip material has a tensile strength of at least 400 psi (2758 kPa).

19. The apparatus of claim 14, wherein the soft tip is a thermoplastic elastomer.

20. The apparatus of claim 14, wherein the soft tip is a silicone.

21. The apparatus of claim 14, wherein the soft tip has a more rigid insert embedded therein which removably couples to a distal end of the push rod.

22. A method for inserting an intraocular lens into an eye, comprising:
forming an incision in a patient's cornea or sclera of no more than 2.2 mm;
providing a cartridge with a load chamber having an open proximal end and a distal end aligned with an injection tube having a tapered internal lumen that terminates at an open mouth, the mouth having an outside diameter of no more than 2.0 mm;
placing an intraocular lens in the load chamber;
inserting a soft tip associated with a push rod into the proximal end of the cartridge;
inserting the open mouth of the injection tube through the incision; and
urging the intraocular lens from the load chamber through the tapered internal lumen, and out of the open mouth of the injection tube by pushing the intraocular lens in a distal direction using the push rod and soft tip.

23. The method of claim 22, wherein the mouth has an outside diameter of no more than 1.8 mm.

24. The method of claim 22, wherein the soft tip is an inflatable member.

25. The method of claim 22, wherein the soft tip is solid.

26. The method of claim 25, wherein the material of the soft tip has an elastic modulus of between 100 psi (689 kPa) and 310 psi (2137 kPa) at an elongation of 100%.

27. The method of claim 25, wherein the material of the soft tip has an ultimate elongation of at least 400%.

28. The method of claim 27, wherein the material of the soft tip is a thermoplastic elastomer.

29. An apparatus for inserting an intraocular lens into an eye, comprising:
a cartridge with a load chamber for receiving an intraocular lens, the load chamber having an open proximal end and a distal end aligned with an injection tube having a tapered internal lumen that terminates at an open mouth, the mouth having an outside diameter of no more than 2.0 mm; and
a push rod having an inflated soft tip associated therewith, the push rod and the cartridge being aligned such that, under urging of the push rod, the soft tip enters the proximal end of the load chamber and pushes distally on an intraocular lens therein.

30. The apparatus of claim 29, wherein the soft tip and push rod are not coupled together.

31. The apparatus of claim 29, wherein the soft tip is underinflated so as to deform as it travels through the tapered internal lumen of the injection tube.
